(19) United States
(12) Patent Application Publication
(10) Pub. No.: US 2026/0182884 A1
(43) Pub. Date: Jul. 2, 2026

(54) TRANSPARENT AND FLEXIBLE BILAYER BIOELECTRODE AND METHOD FOR MANUFACTURING THE SAME AND ELECTRODE SENSOR INCLUDING THE SAME

(71) Applicant: UNIVERSITY OF SEOUL INDUSTRY COOPERATION FOUNDATION, Seoul (KR)

(72) Inventors: Dong Wook PARK, Seongnam-si (KR); Soo Kyeong KIM, Seongnam-si (KR); Hyung Jun CHOI, Seoul (KR)

(21) Appl. No.: 19/045,995

(22) Filed: Feb. 5, 2025

(30) Foreign Application Priority Data
Dec. 30, 2024 (KR) 10-2024-0200870

(51) Int. Cl. A61B 5/268; A61B 5/145; A61B 5/28; A61B 5/291; A61B 5/296

(52) U.S. Cl. CPC A61B 5/268; A61B 5/14546; A61B 5/28; A61B 5/291; A61B 5/296; A61B 2560/0468; A61B 2562/125

(57) ABSTRACT

A bioelectrode is proposed. The bioelectrode may include a substrate and an electrode part formed on the substrate. The electrode part may include a channel having a bilayer structure of a first channel including a first material and a second channel including a second material different from the first material. The first material and the second material may include a biocompatible material.

### CROSS-REFERENCE TO RELATED APPLICATION

This application claims priority to and the benefit of Korean Patent Application No. 10-2024-0200870 filed on Dec. 30, 2024 in the Korean Intellectual Property Office, the entire contents of which are incorporated herein by reference.

### BACKGROUND

### Technical Field

The present disclosure relates to a transparent and flexible bilayer bioelectrode, a method for manufacturing the same, and an electrode sensor including the same.

More specifically, the present disclosure relates to a bilayer bioelectrode, a method for manufacturing the same, and an electrode sensor including the same, which can secure high electrical performance and reliability by applying graphene having excellent electrical conductivity and PEDOT:PSS (poly(3,4-ethylenedioxythiophene) polystyrene sulfonate) effectively lowering electrochemical impedance in a bilayer structure, and which can maximize the usability as a biosensor for detecting a target molecule (for example, cortisol molecule) by fixing a bioreceptor (for example, aptamer) to the bioelectrode or measuring EMG signals and the like by linking with a measuring device.

### Description of Related Technology

Bioelectrode and biosensor technology is a field that is expected to continue to grow as it shows high demand in various fields such as medicine, pharmaceuticals, and environmental monitoring.

When manufacturing such bioelectrodes, biocompatible materials are often used in consideration of bio-application fields. However, if the detailed constituent materials of the bioelectrode are composed only considering biocompatibility, problems such as reduced transmittance and/or flexibility may occur. Accordingly, many parts may be covered by the bioelectrode element, or problems related to physical contact between the human body and the bioelectrode may occur.

### SUMMARY

One aspect is a bilayer bioelectrode, a method for manufacturing the same, and an electrode sensor including the same, which can secure high electrical performance and reliability by applying graphene with excellent electrical conductivity and PEDOT:PSS (poly(3,4-ethylenedioxythiophene) polystyrene sulfonate) that effectively lowers electrochemical impedance in a bilayer structure.

Another aspect is a bilayer bioelectrode, a method for manufacturing the same, and an electrode sensor including the same, which can provide insight into a more suitable bioelectrode structure depending on the sensing environment by comparing and analyzing the structural characteristics by changing the material positions of graphene and PEDOT:PSS in the bioelectrode.

Another aspect is a bilayer bioelectrode and a method for manufacturing the same, and an electrode sensor including the same, which can secure transmittance, flexibility, and biocompatibility by using parylene-C, graphene, and PEDOT:PSS as constituent materials.

Another aspect is a bilayer bioelectrode, a method for manufacturing the same, and an electrode sensor including the same, which can maximize its usability as a biosensor by fixing a bioreceptor (for example, an aptamer) that can bind to a target molecule (for example, a cortisol molecule) to a bioelectrode composed of parylene-C, graphene, and PEDOT:PSS as constituent materials.

Another aspect is a bilayer bioelectrode, a method for manufacturing the same, and an electrode sensor including the same, which can maximize its usability as a biosensor by linking a bioelectrode composed of parylene-C, graphene, and PEDOT:PSS with a measuring device to measure EMG signals. The objects of the present disclosure are not limited to those mentioned above, and other objects and advantages not mentioned can be understood through the following description and will be more clearly understood through the embodiments of the present disclosure. Furthermore, the objects and advantages of the present disclosure can be realized through the means and combinations of those means presented in the claims.

The aspects of the present disclosure are not limited to those mentioned above, and other aspects and advantages not mentioned can be understood through the following description and will be more clearly understood through the embodiments of the present disclosure. Furthermore, the objects and advantages of the present disclosure can be realized through the means and combinations of those means presented in the claims.

Another aspect is a bioelectrode, comprising: a substrate, and an electrode part formed on the substrate, wherein the electrode part includes a channel having a bilayer structure of a first channel including a first material and a second channel including a second material different from the first material, and the first material and the second material include a biocompatible material.

According to some aspects, the first material includes PEDOT:PSS (poly(3,4-ethylenedioxythiophene) polystyrene sulfonate), and the second material includes graphene.

According to some aspects, the first channel is formed on the substrate, the second channel is formed on the first channel, and the first channel is disposed between the substrate and the second channel.

According to some aspects, the second channel is formed on the substrate, the first channel is formed on the second channel, and the second channel is disposed between the substrate and the first channel.

According to some aspects, the constituent material of the substrate includes parylene-C.

Another aspect is an electrode sensor, comprising: a substrate; an electrode part formed on the substrate, and a bio-receptor formed on the electrode part, wherein the electrode part includes a channel having a bilayer structure of a first channel including a first material and a second channel including a second material different from the first material, and the first material and the second material include a biocompatible material.

According to some aspects, the bio-receptor includes an aptamer capable of binding to a predefined target molecule.

According to some aspects, the target molecule includes a cortisol molecule.

According to some aspects, the bio-receptor is fixed to the channel of the electrode part.

Another aspect is an electrode sensor, comprising: a bioelectrode; and a measuring device that is linked to the bioelectrode and constantly measures a biosignal based on a sensing value from the bioelectrode, wherein the bioelectrode includes: a substrate; and an electrode part formed on the substrate, and wherein the electrode part includes a channel having a bilayer structure of a first channel including a first material and a second channel including a second material different from the first material, the first material and the second material include a biocompatible material, and the biosignal includes at least one of electrocardiography (ECG), electroencephalography (EEG), and electromyography (EMG).

The bilayer bioelectrode, the method for manufacturing the same, and the electrode sensor including the same according to some embodiments of the present disclosure can secure high electrical performance and reliability by applying graphene having excellent electrical conductivity and PEDOT:PSS (poly(3,4-ethylenedioxythiophene) polystyrene sulfonate) effectively lowering electrochemical impedance in a bilayer structure.

In addition, the bilayer bioelectrode, the method for manufacturing the same, and the electrode sensor including the same according to some embodiments of the present disclosure can provide insight into a more suitable bioelectrode structure depending on the sensing environment by comparing and analyzing the structural characteristics by changing the material positions of graphene and PEDOT:PSS in the bioelectrode. That is, the bilayer bioelectrode, the method for manufacturing the same, and the electrode sensor including the same according to some embodiments of the present disclosure can provide information on which structure of the bioelectrode is more suitable depending on the target sensor type (for example, EMG sensor (electromyography sensor), ECG sensor (electrocardiography sensor), Aptamer sensor, antigen-antibody sensor (immunosensor), and the like) by comparing and analyzing the structural characteristics by changing the material positions of graphene and PEDOT:PSS in the bioelectrode.

In addition, the bilayer bioelectrode, the method for manufacturing the same, and the electrode sensor including the same according to some embodiments of the present disclosure can secure transmittance, flexibility, and biocompatibility by using parylene-C, graphene, and PEDOT:PSS as constituent materials.

In addition, the bilayer bioelectrode, the method for manufacturing the same, and the electrode sensor including the same according to some embodiments of the present disclosure can maximize the usability as a biosensor by fixing a bioreceptor (for example, aptamer) that can bind to a target molecule (for example, cortisol molecule) to a bioelectrode composed of parylene-C, graphene, and PEDOT:PSS as constituent materials.

In addition, the bilayer bioelectrode, the method for manufacturing the same, and the electrode sensor including the same according to some embodiments of the present disclosure can secure high electrical characteristics, so that in addition to the aptamer sensor described above, there is a high possibility of being used as various biosensors such as an EMG sensor, an ECG sensor, and an antigen-antibody sensor.

In addition to the aforementioned, the specific effects of the invention will be described in detail while explaining the specific aspects of implementing the invention.

### BRIEF DESCRIPTION OF THE DRAWINGS

FIG. 1 is a perspective view of a bioelectrode according to some embodiments of the present disclosure.

FIG. 2 is a front view of a bioelectrode according to some embodiments of the present disclosure.

FIG. 3A and FIG. 3B illustrate a first bioelectrode and a manufacturing method thereof according to some embodiments of the present disclosure.

FIGS. 4A and 4B illustrate a second bioelectrode and a manufacturing method thereof according to some embodiments of the present disclosure.

FIGS. 5A to 5E illustrate experimental data regarding a first bioelectrode and a second bioelectrode according to some embodiments of the present disclosure and a bioelectrode in the prior art.

FIG. 6 illustrates an electrode sensor according to some embodiments of the present disclosure.

FIGS. 7A to 7C illustrate experimental data regarding an electrode sensor according to some embodiments of the present disclosure.

FIG. 8 illustrates an electrode sensor according to some other embodiments of the present disclosure.

FIG. 9 illustrates experimental data regarding the electrode sensor of FIG. 8.

### DETAILED DESCRIPTION

Commercialized bioelectrodes are often made of opaque metals such as gold and platinum, and the opacity of these metals reduces their utility in the field of medical imaging. In other words, if a bioelectrode is made only of constituent materials that do not ensure transmittance, there is a problem in that changes in the measurement part cannot be observed in real time while measuring and stimulating biosignals due to the opacity.

To overcome this, many studies are being conducted on bioelectrodes that are both biocompatible and transparent, and electrode sensors using these bioelectrodes.

The terms or words used in the disclosure and the claims should not be construed as limited to their ordinary or lexical meanings. They should be construed as the meaning and concept in line with the technical idea of the disclosure based on the principle that the inventor can define the concept of terms or words in order to describe his/her own inventive concept in the best possible way. Further, since the embodiment described herein and the configurations illustrated in the drawings are merely one embodiment in which the disclosure is realized and do not represent all the technical ideas of the disclosure, it should be understood that there may be various equivalents, variations, and applicable examples that can replace them at the time of filing this application.

Although terms such as first, second, A, B, etc. used in the description and the claims may be used to describe various components, the components should not be limited by these terms. These terms are only used to differentiate one component from another. For example, a first component may be referred to as a second component, and similarly, a second component may be referred to as a first component, without departing from the scope of the disclosure. The term ‘and/or’ includes a combination of a plurality of related listed items or any item of the plurality of related listed items.

The terms used in the description and the claims are merely used to describe particular embodiments and are not intended to limit the disclosure. Singular forms are intended to include plural forms unless the context clearly indicates otherwise. In the application, terms such as “comprise,” “comprise,” “have,” etc. should be understood as not precluding the possibility of existence or addition of features, numbers, steps, operations, components, parts, or combinations thereof described herein.

Unless otherwise defined, the phrases “A, B, or C,” “at least one of A, B, or C,” or “at least one of A, B, and C” may refer to only A, only B, only C, both A and B, both A and C, both B and C, all of A, B, and C, or any combination thereof.

Unless being defined otherwise, all terms used herein, including technical or scientific terms, have the same meaning as commonly understood by those skilled in the art to which the disclosure pertains.

Terms such as those defined in commonly used dictionaries should be construed as having a meaning consistent with the meaning in the context of the relevant art, and are not to be construed in an ideal or excessively formal sense unless explicitly defined in the application. In addition, each configuration, procedure, process, method, or the like included in each embodiment of the disclosure may be shared to the extent that they are not technically contradictory to each other.

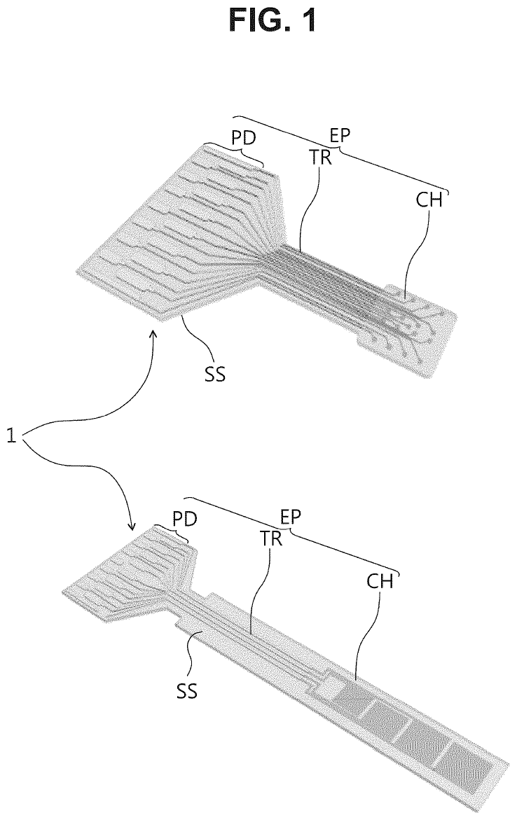

Hereinafter, with reference to FIGS. 1 to 9, a bilayer bioelectrode, a method for manufacturing the same, and an electrode sensor including the same according to some embodiments of the present disclosure will be described in detail.

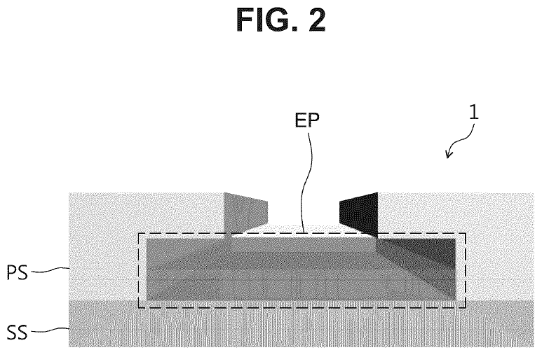

FIG. 1 is a perspective view of a bioelectrode according to some embodiments of the present disclosure. FIG. 2 is a front view of a bioelectrode according to some embodiments of the present disclosure.

Referring to FIGS. 1 and 2, a biological electrode (hereinafter referred to as “BE”) according to some embodiments of the present disclosure may include a substrate (hereinafter referred to as “SS”), an electrode part (hereinafter referred to as “EP”), and a passivation (hereinafter referred to as “PS”).

The substrate (SS) may serve as the base or foundation of the biological electrode (BE). In some examples, the substrate (SS) may include parylene-C. In other words, the constituent material of the substrate (SS) may include parylene-C. However, the embodiment of the present disclosure is not limited thereto, and the constituent material of the substrate (SS) may include polyimide (PI) and polydimethylsiloxane (PDMS). Hereinafter, for convenience of explanation, it will be described assuming that the constituent material of the substrate (SS) is parylene-C.

In some examples, the substrate (SS) may be formed by coating parylene-C on a semiconductor wafer. In this case, parylene-C may be coated on the semiconductor wafer through a chemical vapor deposition (CVD) method. However, the embodiment of the present disclosure is not limited thereto, and parylene-C may be coated on the semiconductor wafer through a physical vapor deposition (PVD) method and/or a plating method.

The electrode part (EP) may include a channel (hereinafter referred to as “CN”), a trace (hereinafter referred to as “TR”), and a pad (hereinafter referred to as “PD”).

The channel (CH) may measure measurement data about the user's body. In some examples, the channel (CH) may contact a body part of the user's body that is to be measured or an electrolyte area to cause an electrochemical reaction, thereby measuring measurement data.

The trace (TR) is connected to the channel (CH) and may transmit measurement data measured by the channel (CH) to the pad (PD). In other words, the trace (TR) may play a role of connecting the channel (CH) and the pad (PD).

The pad (PD) is connected to the trace (TR) and may receive measurement data from the trace (TR) and output it to the outside. In some examples, the pad (PD) may output the measurement data transmitted through the trace (TR) to an external element such as a measurement device or a printed circuit board (PCB).

The passivation (PS) may play a role of protecting the bioelectrode (BE) from oxygen or moisture. In some examples, the constituent material of the passivation (PS) may include a COP (Cyclic Olefin Polymer), but the embodiments of the present disclosure are not limited thereto. In this case, the passivation (PS) may be formed by spin-coating COP on the substrate (SS) and the electrode part (EP).

In some examples, at least one of the channel (CH), trace (TR), and pad (PD) included in the electrode part (EP) may be composed of a material that is biocompatible and flexible, and ensures transmittance.

More specifically, the channel (CH) included in the electrode part (EP) may include at least one of graphene, PEDOT:PSS (poly(3,4-ethylenedioxythiophene)-poly(styrenesulfonate)), and silver nanowires (AgNWs). However, the embodiment of the present disclosure is not limited thereto, and it is obvious that the channel (CH) may include more diverse materials, substances, and components.

Meanwhile, the bioelectrode 1 and/or the electrode part (EP) may have a bilayer structure. In other words, the channel (CH) of the electrode part (EP) included in the bioelectrode 1 according to some embodiments of the present disclosure may have a bilayer structure of a first channel and a second channel. That is, the channel (CH) included in the electrode part (EP) may include a bilayer structure.

In this case, the first channel and the second channel may include different materials. In other words, the channel (CH) of the electrode part (EP) included in the bioelectrode 1 may include a bilayer structure composed of different materials.

For example, the first material constituting the first channel formed on the substrate (SS) in the channel (CH) may include PEDOT:PSS, and the second material constituting the second channel formed on the first channel may include graphene. In other words, the channel (CHa) according to the first embodiment of the present disclosure may include a structure in which PEDOT:PSS is formed on the substrate (SS) and graphene is formed on the PEDOT:PSS.

In another example, the first material constituting the first channel formed on the substrate (SS) in the channel (CH) may include graphene, and the second material constituting the second channel formed on the first channel may include PEDOT:PSS. In other words, the channel (CHb) according to the second embodiment of the present disclosure may include a structure in which graphene is formed on the substrate (SS) and PEDOT:PSS is formed on the graphene.

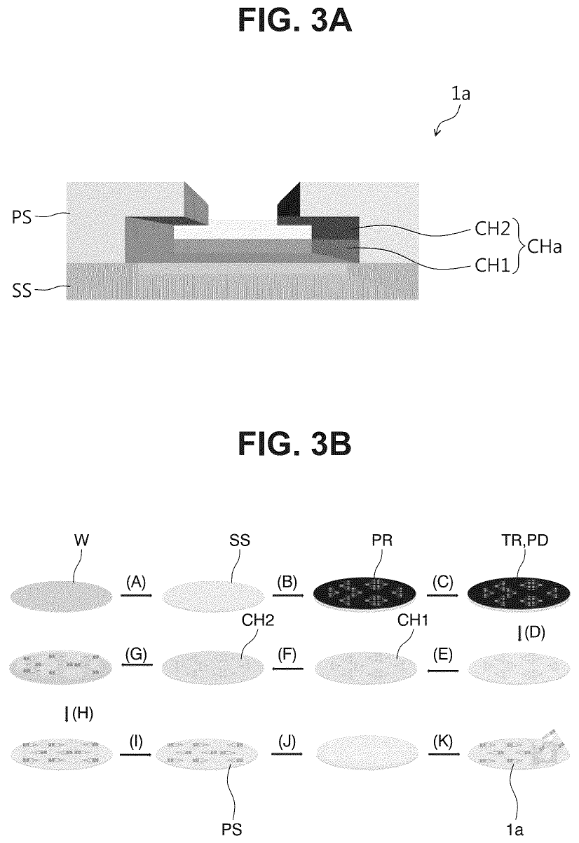

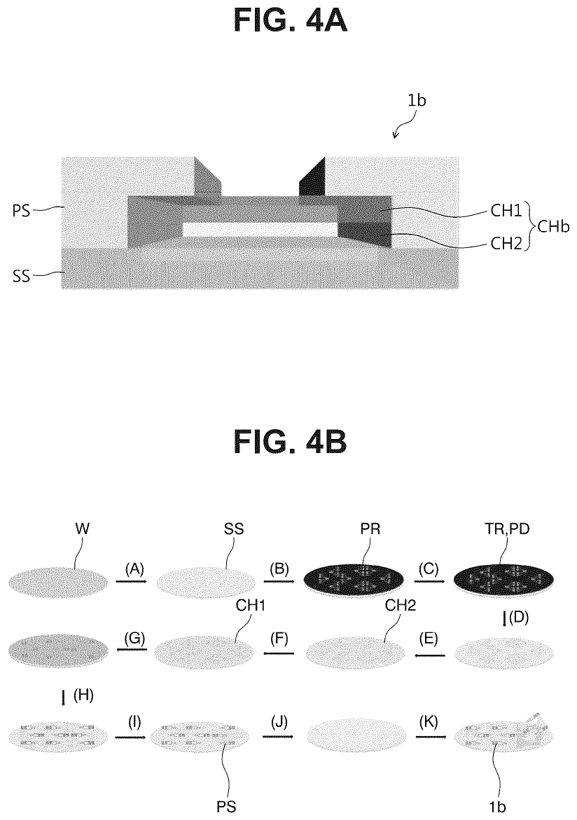

Hereinafter, the channel (CHa) according to the first embodiment of the present disclosure will be described with reference to FIGS. 3A and 3B, and the channel (CHb) according to the second embodiment of the present disclosure will be described with reference to FIGS. 4A and 4B.

FIG. 3A and FIG. 3B illustrate a first bioelectrode and a manufacturing method thereof according to some embodiments of the present disclosure. The manufacturing method of FIG. 3B may be performed by a separate bioelectrode manufacturing device.

Referring to FIG. 1, FIG. 2, FIG. 3A and FIG. 3B, a first bioelectrode 1a according to some embodiments of the present disclosure may include a substrate (SS), a channel (CHa), and a passivation (PS). The substrate (SS) and the passivation (PS) have been described in detail with reference to FIG. 1 and FIG. 2, and are therefore omitted here.

The first bioelectrode 1a according to some embodiments of the present disclosure may have a bilayer structure. In other words, a channel (CHa) included in the first bioelectrode 1a according to some embodiments of the present disclosure may have a bilayer structure of a first channel CH1 and a second channel CH2.

In this case, the first channel CH1 and the second channel CH2 may include different materials. In other words, the channel (CHa) included in the first bioelectrode 1a may include a bilayer structure composed of different materials.

For example, the first material constituting the first channel CH1 formed on the substrate (SS) in the channel (CHa) may include PEDOT:PSS, and the second material constituting the second channel CH2 formed on the first channel CH1 may include graphene. In other words, the channel (CHa) according to some embodiments of the present disclosure may include a structure in which PEDOT:PSS is formed on the substrate (SS) and graphene is formed on the PEDOT:PSS. Accordingly, the first channel CH1 having PEDOT:PSS as the first material may be placed between the substrate (SS) and the second channel CH2.

The first bioelectrode 1a may be manufactured and fabricated according to a predetermined method and procedure. Hereinafter, the manufacturing process of the first bioelectrode 1a according to some embodiments of the present disclosure will be described in detail.

First, as shown in (A) of FIG. 3B, in order to manufacture and fabricate the first bioelectrode 1a according to some embodiments of the present disclosure, a substrate (SS) may be coated on a wafer (hereinafter referred to as “W”). The wafer (W) may include a 4-inch silicon wafer (SI Wafer), but the embodiments of the present disclosure are not limited thereto. In some examples, the substrate (SS) may be formed by coating parylene-C of a predetermined thickness on the wafer (W). In this case, the substrate (SS) may be formed by coating parylene-C on the wafer (W) through a chemical vapor deposition (CVD) method, such as a thermal chemical vapor deposition (thermal CVD). However, the embodiments of the present disclosure are not limited thereto, and the substrate (SS) may be deposited through other methods, such as a physical vapor deposition (PVD) method or a plating method.

Then, as shown in (B) of FIG. 3B, a photoresist (hereinafter referred to as “PR”) of a specific pattern may be formed on the substrate (SS) through a photolithography process.

Then, as shown in (C) of FIG. 3B, a trace (TR) and a pad (PD) may be formed by coating and depositing a metal on the substrate (SS) on which the photoresist (PR) is formed through an electron beam physical vapor deposition (electron beam PVD). In this case, the metal may include titanium (Ti) and gold (Au), but the embodiment of the present disclosure is not limited thereto.

Then, as shown in (D) of FIG. 3B, the photoresist (PR) may be removed through a lift-off process.

Next, as shown in (E) of FIG. 3B, a first channel CH1 may be formed. In this case, the first material constituting the first channel CH1 may include PEDOT:PSS. In some examples, the first channel CH1 may be formed by spin-coating PEDOT:PSS on the substrate (SS).

Next, as shown in (F) of FIG. 3B, a second channel CH2 may be formed. In this case, the second material constituting the second channel CH2 may include graphene. In some examples, the second channel CH2 may be formed by transferring graphene onto the first channel CH1.

Next, as shown in (G) of FIG. 3B, an etching mask used in the etching process may be patterned. In some examples, the etching mask may be patterned through a photolithography process.

Then, as shown in (H) of FIG. 3b, dry etching may be performed.

Then, as shown in (I) of FIG. 3B, a passivation (PS) may be formed. In some examples, the passivation (PS) may be formed by spin-coating a COP on at least one of the substrate (SS), the channel (CH), the trace (TR), and the pad (PD), and then opening the contact portion of the pad (PD) and the channel (CH) through a photolithography process. However, the embodiments of the present disclosure are not limited thereto, and the passivation material may include various materials in addition to the COP described above. In addition, the process of forming the passivation (PS) of (I) of FIG. 3B may include a process of forming a COP pattern by performing a photolithography process using a photosensitive material (for example, SU-8). In this case, the photosensitive material (for example, SU-8) may also be patterned using a photolithography process, similar to COP.

Subsequently, as shown in (J) of FIG. 3B, a metal pattern using aluminum (hereinafter referred to as “AL”) as a constituent material may be formed. In other words, for the etching of the substrate (SS) to be performed later, a metal pattern using aluminum (AL) as a constituent material may be formed on the electrode part (EP) and the passivation (PS). In this case, the formation of a photoresist (PR), the deposition of aluminum (AL), and the lift-off process may be performed in sequence.

Then, as shown in (K) of FIG. 3B, the first bioelectrode 1a may be released by etching the substrate (SS) through dry etching.

FIGS. 4A and 4B illustrate a second bioelectrode and a manufacturing method thereof according to some embodiments of the present disclosure. The manufacturing method of FIG. 4B may be performed by a separate bioelectrode manufacturing device.

Referring to FIGS. 1, 2, 4A and 4B, the second bioelectrode 1b according to some embodiments of the present disclosure may include a substrate (SS), a channel (CHb), and a passivation (PS). The substrate (SS) and the passivation (PS) have been described in detail with reference to FIGS. 1 and 2, and are therefore omitted here.

The second bioelectrode 1b according to some embodiments of the present disclosure may have a bilayer structure. In other words, the channel (CHb) included in the second bioelectrode 1b according to some embodiments of the present disclosure may have a bilayer structure of the first channel CH1 and the second channel CH2.

In this case, the first channel CH1 and the second channel CH2 may include different materials. In other words, the channel (CHb) included in the second bioelectrode 1b may include a bilayer structure composed of different materials.

For example, the second material constituting the second channel CH2 formed on the substrate (SS) in the channel (CHb) may include graphene, and the first material constituting the first channel CH1 formed on the second channel CH2 may include PEDOT:PSS. In other words, the channel (CHb) according to some embodiments of the present disclosure may include a structure in which graphene is formed on the substrate (SS) and PEDOT:PSS is formed on the graphene. Accordingly, the second channel CH2 having graphene as a second material may be placed between the substrate (SS) and the first channel CH1.

The second bioelectrode 1b may be manufactured and fabricated according to a predetermined method and procedure. Hereinafter, a manufacturing process of the second bioelectrode 1b according to some embodiments of the present disclosure will be described in detail.

First, as shown in (A) of FIG. 4B, in order to manufacture and fabricate the second bioelectrode 1b according to some embodiments of the present disclosure, a substrate (SS) may be coated on a wafer (W). The wafer (W) may include a 4-inch silicon wafer (SI Wafer), but the embodiments of the present disclosure are not limited thereto. In some examples, the substrate (SS) may be formed by coating parylene C of a predetermined thickness on the wafer (W). In this case, the substrate (SS) may be formed by coating parylene C on the wafer (W) through a chemical vapor deposition (CVD) method, such as a thermal chemical vapor deposition (thermal CVD). However, the embodiment of the present disclosure is not limited thereto, and the substrate (SS) may be deposited through other methods such as physical vapor deposition (PVD) or plating.

Subsequently, as shown in (B) of FIG. 4B, a photoresist (hereinafter referred to as “PR”) of a specific pattern may be formed on the substrate (SS) through a photolithography process.

Subsequently, as shown in (C) of FIG. 4B, a trace (TR) and a pad (PD) may be formed by coating and depositing a metal on the substrate (SS) on which the photoresist (PR) is formed through an electron beam PVD process. In this case, the metal may include titanium (Ti) and gold (Au), but the embodiment of the present disclosure is not limited thereto.

Then, as shown in (D) of FIG. 4b, the photoresist (PR) may be removed through a lift-off process.

Then, as shown in (E) of FIG. 4B, a second channel CH2 may be formed. In this case, the second material constituting the second channel CH2 may include graphene. In some examples, the second channel CH2 may be formed by transferring graphene onto the substrate (SS).

Then, as shown in (F) of FIG. 4B, a first channel CH1 may be formed. In this case, the first material constituting the first channel CH1 may include PEDOT:PSS. In some examples, the first channel CH1 may be formed by spin-coating PEDOT:PSS on the second channel CH2.

Next, as shown in (G) of FIG. 4B, an etching mask used in an etching process may be patterned. In some examples, the etching mask may be patterned through a photolithography process.

Next, as shown in (H) of FIG. 4B, dry etching may be performed.

Next, as shown in (I) of FIG. 4B, a passivation (PS) may be formed. In some examples, the passivation (PS) may be formed by spin-coating a COP on at least one of a substrate (SS), a channel (CH), a trace (TR), and a pad (PD), and then opening a contact portion of the pad (PD) and the channel (CH) through a photolithography process. However, the embodiments of the present disclosure are not limited thereto, and the passivation material may include various materials in addition to the above-described COP. In addition, the process of forming the passivation (PS) of (I) of FIG. 3B may include a process of forming a COP pattern by performing a photolithography process using a photosensitive material (for example, SU-8). In this case, the photosensitive material (for example, SU-8) may also be patterned using a photolithography process, similar to COP.

Subsequently, as shown in (J) of FIG. 4B, a metal pattern using aluminum (hereinafter referred to as “AL”) as a constituent material may be formed. In other words, for the etching of the substrate (SS) to be performed later, a metal pattern using aluminum (AL) as a constituent material may be formed on the electrode part (EP) and the passivation (PS). In this case, the formation of a photoresist (PR), the deposition of aluminum (AL), and the lift-off process may be performed in sequence.

Then, as shown in (K) of FIG. 4B, the second bioelectrode 1b may be released by etching the substrate (SS) through dry etching.

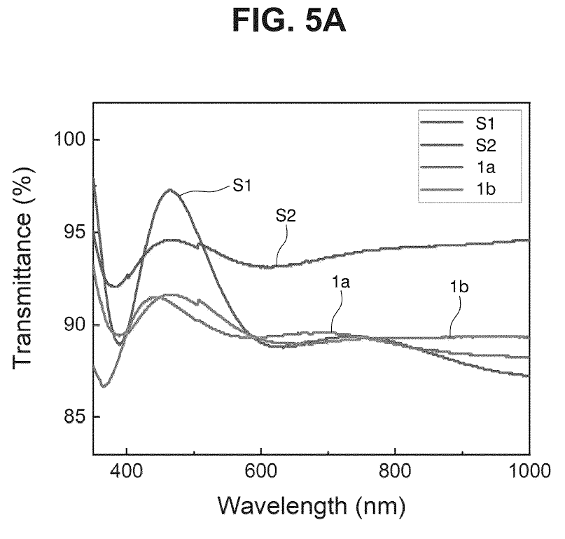

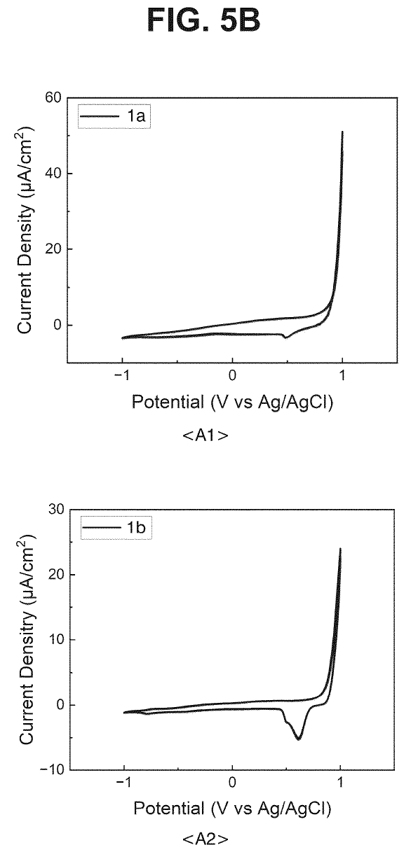

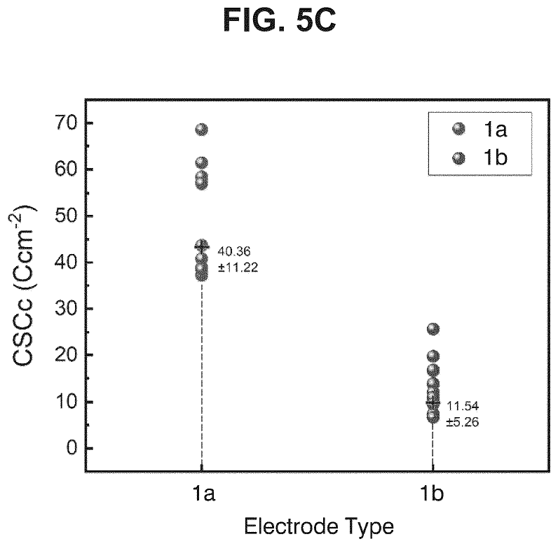

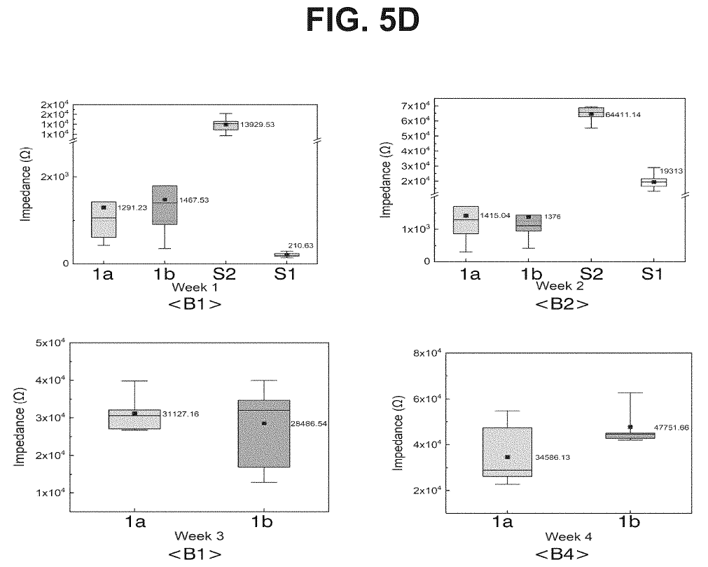

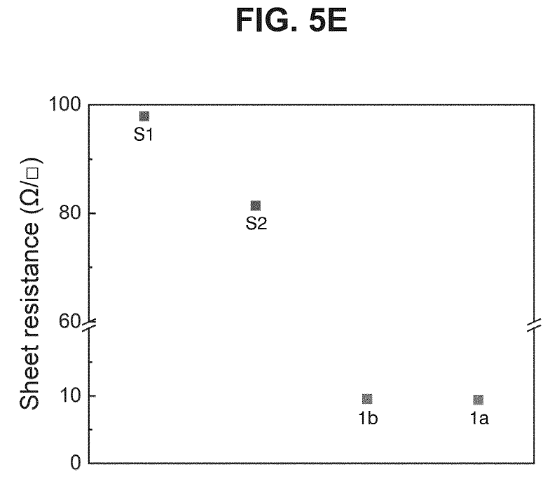

Hereinafter, experimental data regarding the first bioelectrode 1a and the second bioelectrode 1b according to some embodiments of the present disclosure will be described with reference to FIGS. 5A to 5E.

FIGS. 5A to 5E illustrate experimental data regarding a first bioelectrode and a second bioelectrode according to some embodiments of the present disclosure and a bioelectrode in the prior art. In other words, the experimental data illustrated in FIGS. 5A to 5E are experimental data regarding the first bioelectrode 1a, the second bioelectrode 1b, a first conventional bioelectrode S1 having a single-layer structure made of PEDOT:PSS rather than a bilayer structure, and a second conventional bioelectrode S2 having a single-layer structure made of graphene rather than a bilayer structure.

First, referring to FIG. 5A, FIG. 5A shows the results of an experiment on the transmittance according to the wavelength of each of the first bioelectrode 1a, the second bioelectrode 1b, the first conventional bioelectrode S1, and the second conventional bioelectrode S2. As shown in FIG. 5A, in the wavelength range of visible light, the first bioelectrode 1a and the second bioelectrode 1b have a transmittance of approximately 90%. This suggests that the first bioelectrode 1a and the second bioelectrode 1b do not cause defects or generate artifacts in the biological environment.

Next, referring to FIG. 5B, FIG. 5B shows the results of an experiment on cyclic voltammetry (CV) to measure the electrochemical capacitance of each of the first bioelectrode 1a and the second bioelectrode 1b. The horizontal axis illustrated in FIG. 5B represents potential, and the vertical axis represents current density. As illustrated in FIG. 5B, the first bioelectrode 1a and the second bioelectrode 1b exhibit high current density, and it can be seen that the electrochemical performance is superior when compared to single-layer channels such as the first conventional bioelectrode S1 and the second conventional bioelectrode S2.

Next, referring to FIG. 5C, FIG. 5C shows the results of an experiment on cathodic charge storage capacitance (CSCc) in each of the first bioelectrode 1a and the second bioelectrode 1b. The horizontal axis illustrated in FIG. 5C represents the electrode type, and the vertical axis represents the measured value for CSCc. In this case, CSCc represents the capacity that indicates the ability of the electrode to store charge within a specific potential range. In addition, CSCc contributes to signal amplification and sensitivity enhancement in biosensors, and the charge storage capacity on the electrode surface is related to the accuracy of the analysis signal. As shown in FIG. 5C, it can be seen that the first bioelectrode 1a has a very high CSCc value, and the second bioelectrode 1b also has a high CSCc value. In contrast, the electrode having a channel formed of metal had a CSCc value of 26.56, and the second conventional bioelectrode S2 having a single-layer structure made of graphene had a CSCc value of 12.66. In particular, it can be seen that the first bioelectrode 1a has a very high CSCc value compared to these conventional electrodes.

Next, referring to FIG. 5D, FIG. 5D shows the results of an experiment on impedance according to the time elapsed (Week1, Week2, Week3, Week4) after manufacturing of each of the first bioelectrode 1a, the second bioelectrode 1b, the first conventional bioelectrode S1, and the second conventional bioelectrode S2. In this case, the impedance was measured by electrochemical impedance spectroscopy (EIS). As shown in FIG. 5D, the first bioelectrode 1a and the second bioelectrode 1b show lower impedance levels than the first conventional bioelectrode S1 and the second conventional bioelectrode S2. The effect of impedance on the signal-to-noise ratio (SNR) is related to the reliability of the device, and it can be seen that the first bioelectrode 1a and the second bioelectrode 1b have low impedance, making them very suitable for bioelectrodes and in-vivo electrode sensors.

Next, referring to FIG. 5E, FIG. 5E shows the experimental results of sheet resistance of the first bioelectrode 1a, the second bioelectrode 1b, the first conventional bioelectrode S1, and the second conventional bioelectrode S2, respectively. The sheet resistances of the first bioelectrode 1a, the second bioelectrode 1b, the first conventional bioelectrode S1, and the second conventional bioelectrode S2, respectively, were measured as 9.43, 9.55, 97.87, and 81.37, respectively. Accordingly, it can be seen that the first bioelectrode 1a and the second bioelectrode 1b have lower surface resistance and higher electrical characteristics and electrical conductivity than the first conventional bioelectrode S1 and the second conventional bioelectrode S2.

Meanwhile, the bioelectrode 1 according to some embodiments of the present disclosure can be utilized as an externally attached sensor such as a wearable sensor, an EMG (electromyography) sensor, or various internal nerve electrodes used for DBS (deep brain stimulation), ECoG (electrocorticograhy), and the like.

Hereinafter, electrode sensors including the bioelectrode 1 according to some embodiments of the present disclosure will be described.

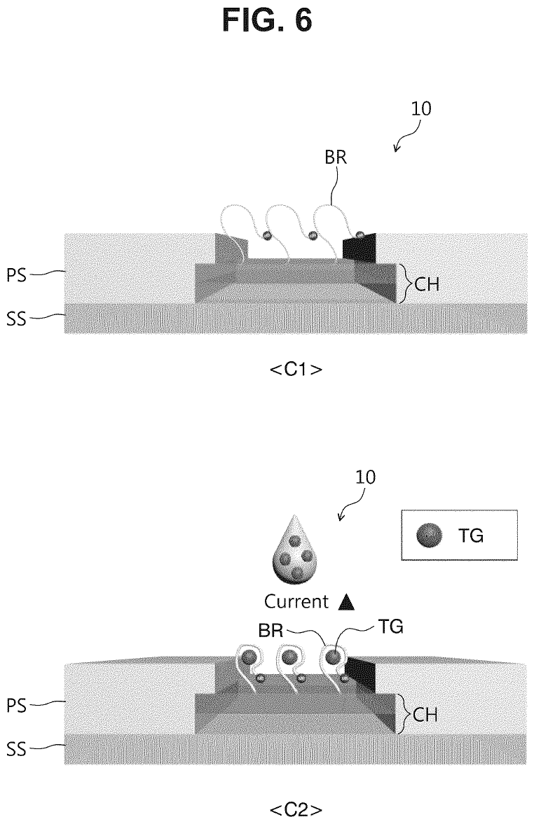

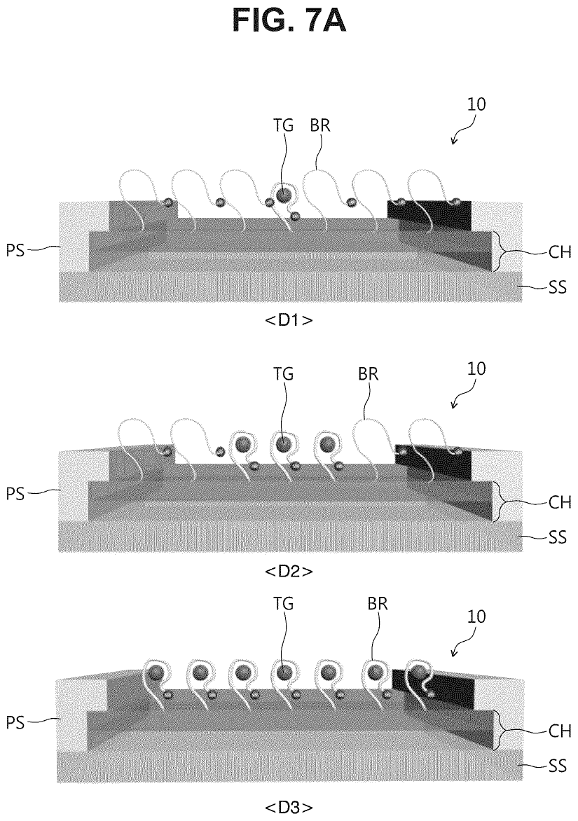

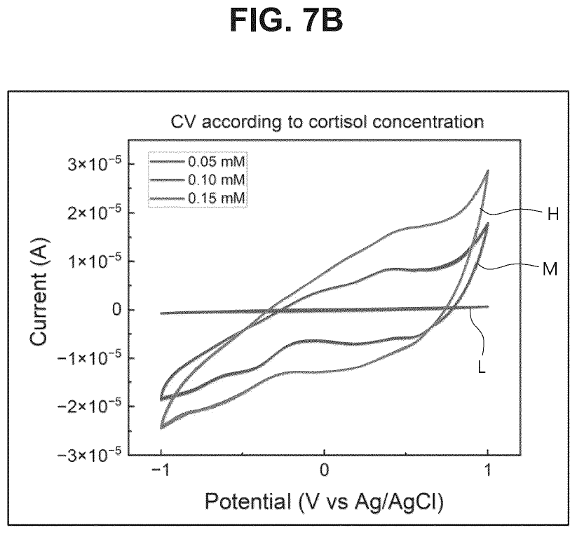

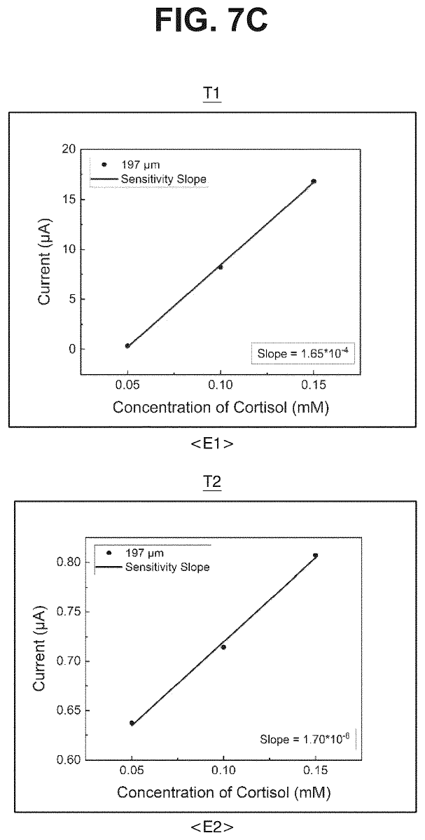

FIG. 6 illustrates an electrode sensor according to some embodiments of the present disclosure. FIGS. 7A to 7C illustrate experimental data regarding an electrode sensor according to some embodiments of the present disclosure.

Referring to FIGS. 1 to 4B and FIG. 6, the electrode sensor 10 according to some embodiments of the present disclosure may further include a bioreceptor (hereinafter referred to as “BR”) in addition to the bioelectrode 1 described above in FIG. 1. In other words, the electrode sensor 10 according to some embodiments of the present disclosure may include a first bioelectrode 1a and a bioreceptor (BR) described above in FIGS. 3A and 3B, and an electrode sensor 10 according to other embodiments of the present disclosure may include a second bioelectrode 1b and a bioreceptor (BR) described above in FIGS. 4A and 4B.

The bioreceptor (BR) may include an aptamer capable of binding to a predefined target molecule (hereinafter referred to as “TG”). However, the embodiment of the present disclosure is not limited thereto. For example, the aptamer may bind to a cortisol molecule. In other words, the target molecule (TG) may include a cortisol molecule, and the aptamer may include a cortisol aptamer. However, the embodiment of the present disclosure is not limited thereto, and the aptamer may bind to other biomolecules.

The cortisol aptamer may include a single nano-rod DNA structure. In this case, the cortisol aptamer may include a thiol group at the bottom and methylene blue having a positive charge at the top. In this case, the thiol group at the bottom of the cortisol aptamer is attached to the channel (CH), and the methylene blue at the top of the cortisol aptamer may bind to cortisol through interaction. In this case, the binding between the cortisol aptamer and cortisol may include non-covalent binding (for example, hydrogen bonding, hydrophobic interaction, electrostatic interaction, conformational fit, and the like). In this case, as the DNA bends, the phase change of methylene blue occurs, and a potential difference occurs accordingly, and the cortisol aptamer can measure the target molecule, (TG), for example, a hormone (cortisol) by using this.

Meanwhile, the aptamer may be attached, fixed, installed, and formed on the channel (CH). In other words, the bioreceptor (BR) including the aptamer may be attached, fixed, installed, and formed in the channel (CH) of the bioelectrode 1 included in the electrode sensor 10.

In this case, the method of attaching the aptamer to the channel (CH) of the electrode sensor 10 may differ depending on the type of the bioelectrode 1. In other words, the presence or absence of an adhesion promoter involved in the attachment of the aptamer and the channel (CH) of the electrode sensor 10 may differ depending on the type of the bioelectrode 1.

For example, when the electrode sensor 10 includes the first bioelectrode 1a described above in FIGS. 3A and 3B, the electrode sensor 10 may be manufactured by attaching the graphene forming the second channel CH2 of the first bioelectrode 1a and the aptamer through the adhesion promoter. In other words, when the electrode sensor 10 includes the first bioelectrode 1a described above in FIGS. 3A and 3B, the electrode sensor 10 may include an attachment promoter as a component.

In another example, when the electrode sensor 10 includes the second bioelectrode 1b described above in FIGS. 4A and 4B, the electrode sensor 10 may not include an attachment promoter. In this case, the PEDOT:PSS constituting the first channel (CH) of the second bioelectrode 1b and the aptamer may be directly bonded. This is because PEDOT:PSS has hydrophilicity itself, and its hydrophilicity is increased through GOPS (glycidoxypropyltrimethoxysilane) and DMSO (dimethyl sulfoxide) used for doping. In particular, GOPS acts as an epoxy-based cross-linker to form a covalent bond with the thiol group at the bottom of the aptamer. That is, as the surface structure of PEDOT:PSS and the arrangement of PSS are changed by DMSO, the active site where GOPS and thiol groups may react increases. Additionally, since the silane group of GOPS provides an additional reaction site on the surface of PEDOT:PSS, the first channel (CH) of the second bioelectrode 1b and the thiol group at the bottom of the aptamer may be attached more easily.

Hereinafter, experimental data for the electrode sensor 10 according to some embodiments of the present disclosure will be described with further reference to FIGS. 7A to 7C. FIGS. 7A to 7C are experimental results obtained through the electrode sensor 10 including the second bioelectrode 1b and the bioreceptor (BR) described above in FIGS. 4A and 4B.

Referring to FIGS. 1 to 4B and FIGS. 6 to 7C, FIG. 7A illustrates an experiment conducted with different concentrations of target molecules (TG), FIG. 7B illustrates an experiment conducted with CV according to the concentration of target molecules (TG), and FIG. 7C illustrates a current measured over time according to the concentration of target molecules (TG).

First, <D1> of FIG. 7A illustrates a case where the concentration of target molecules (TG) is low concentration (hereinafter referred to as “L”), and accordingly, FIG. 7B illustrates a CV graph at low concentration (L). In this case, the low concentration (L) was set to 0.05 mM. In addition, <D2> of FIG. 7A illustrates a case where the concentration of the target molecule (TG) is a middle concentration (hereinafter referred to as “M”), and accordingly, a CV graph at the middle concentration (M) is illustrated in FIG. 7B. In this case, the middle concentration (M) was set to 0.10 mM. In addition, <D3> of FIG. 7A illustrates a case where the concentration of the target molecule (TG) is a high concentration (hereinafter referred to as “H”), and accordingly, a CV graph at the high concentration (H) is illustrated in FIG. 7B. In this case, the high concentration (H) was set to 0.15 mM.

As shown in FIG. 7B, it can be seen that the current (Concentration) increases as the concentration of the target molecule (TG) increases, and accordingly, it can be seen that the electrode sensor 10 according to some embodiments of the present disclosure may measure the amount of the target molecule (TG) and, for example, may detect cortisol molecules effectively.

<E1> of FIG. 7C shows the current (Current) measured according to the concentration of the target molecule (TG) at the first time point (T1), and <E2> of FIG. 7C shows the current measured according to the concentration of the target molecule (TG) at the second time point (T2). In this case, the first time point (T1) is the time point when the electrode sensor 10 was manufactured, and the second time point (T2) is the time point 7 days after the electrode sensor 10 was manufactured.

As shown in FIG. 7C, when the current according to the concentration of the target molecule (TG) was measured with the same electrode sensor 10 at the second time point (T2) approximately 7 days after the first time point (T1), the absolute value of the current was measured to be relatively decreased compared to the first time point (T1). However, it was found that the current value according to the concentration of the target molecule (TG) still showed a linear tendency. Accordingly, it was found that the target molecule (TG) could be detected for at least 7 days.

Meanwhile, it may be more reasonable for the electrode sensor 10 for cortisol detection according to some embodiments of the present disclosure to include the second bioelectrode 1b described in FIGS. 4A and 4B rather than the first bioelectrode 1a described in FIGS. 3A and 3B. In other words, the electrode sensor 10 for cortisol detection according to some embodiments of the present disclosure may include the second bioelectrode 1b and the bioreceptor (BR) described in FIGS. 4A and 4B for better performance. This is because the second bioelectrode 1b has excellent chemical reactivity, has good compatibility with electrochemical applications, and is therefore more suitable for the electrode sensor 10 of the present disclosure using aptamers.

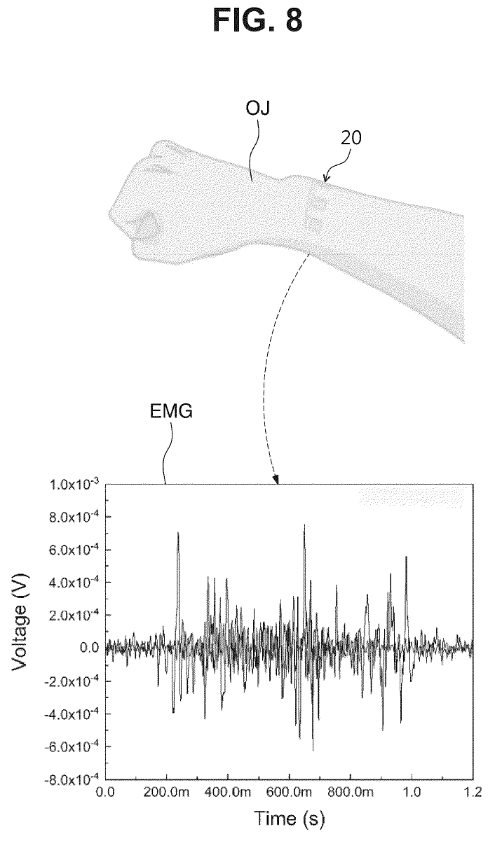

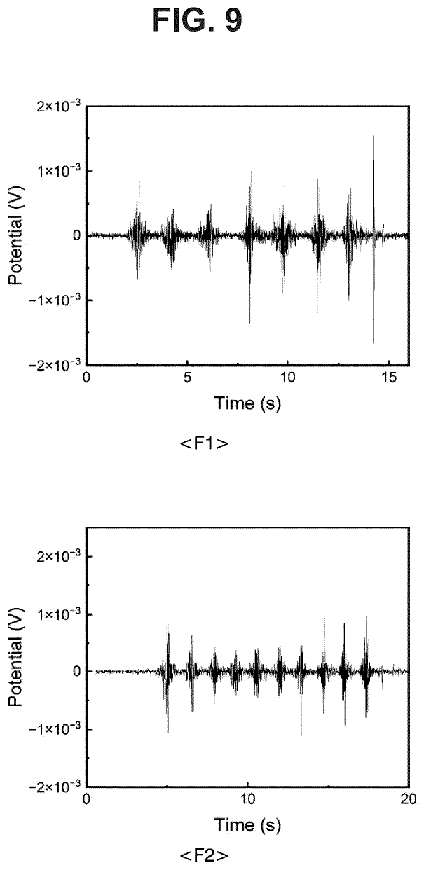

FIG. 8 illustrates an electrode sensor according to some other embodiments of the present disclosure. FIG. 9 illustrates experimental data regarding the electrode sensor of FIG. 8.

Referring to FIGS. 1 to 4B and FIG. 8, the electrode sensor 20 according to some embodiments of the present disclosure may further include a measuring device in addition to the bioelectrode 1 described in FIG. 1. In other words, the electrode sensor 20 may further include the bioelectrode 1 described in FIG. 1 and a measuring device linked to the bioelectrode 1. In this case, the bioelectrode 1 may include either the first bioelectrode 1a described in FIGS. 3A and 3B or the second bioelectrode 1b described in FIGS. 4A and 4B.

The measuring device may be an electronic device that amplifies and detects a biosignal through the sensing value of the bioelectrode 1. For example, the measuring device may measure a biosignal including an electrocardiogram (ECG), an electroencephalogram (EEG), an electromyogram (EMG), and the like based on the sensing value of the channel (CN) of the bioelectrode 1. For convenience of explanation, FIG. 8 illustrates a case where the electrode sensor 20 measures an electromyogram (EMG), but this is only for convenience of explanation.

Meanwhile, referring to FIGS. 1 to 4B, FIGS. 8 and 9, FIG. 9 shows an experiment on potential changes according to a user's fist-clenching or pinching while wearing the electrode sensor 20 according to some embodiments of the present disclosure.

More specifically, <F1> of FIG. 9 is experimental data measured through the electrode sensor 20 including the first bioelectrode 1a described in FIGS. 3A and 3B, and <F2> of FIG. 9 is experimental data measured through the electrode sensor 20 including the second bioelectrode 1b described in FIGS. 4A and 4B.

As shown in <F1> and <F2> of FIG. 9, since a change in potential is observed depending on whether the user clenches or pinches his fist, it can be seen that the electrode sensor 20 according to some embodiments of the present disclosure may be utilized as an electromyography sensor.

In this case, the SNR in <F1> of FIG. 9 was measured as 20.39, and the SNR in <F2> of FIG. 9 was measured as 17.01. Accordingly, it can be seen that the electrode sensor 20 including the first bioelectrode 1a described in FIGS. 3A and 3B may be utilized as an electromyography sensor with higher reliability.

Meanwhile, it may be more appropriate for the electrode sensor 20 for detecting biosignals according to some embodiments of the present disclosure to include the first bioelectrode 1a described in FIGS. 3A and 3B rather than the second bioelectrode 1b described in FIGS. 4A and 4B. In other words, the electrode sensor 20 for detecting biosignals including EMG and the like according to some embodiments of the present disclosure may include the first bioelectrode 1a described in FIGS. 3A and 3B for better performance. This is because the first bioelectrode 1a may have higher electrical characteristics and is therefore more suitable for the electrode sensor 20 of the present disclosure, which must detect a signal in a noisy environment.

While the inventive concept has been particularly shown and described with reference to exemplary embodiments thereof, it will be understood by those of ordinary skill in the art that various changes in form and details may be made therein without departing from the spirit and scope of the inventive concept as defined by the following claims. It is therefore desired that the embodiments be considered in all respects as illustrative and not restrictive, reference being made to the appended claims rather than the foregoing description to indicate the scope of the disclosure.

What is claimed is:

1. A bioelectrode, comprising:
a substrate; and
an electrode part formed on the substrate,
wherein the electrode part includes a channel having a bilayer structure of a first channel including a first material and a second channel including a second material different from the first material, and
wherein the first material and the second material include a biocompatible material.

2. The bioelectrode according to claim 1, wherein:
the first material includes PEDOT:PSS (poly(3,4-ethylenedioxythiophene) polystyrene sulfonate), and
the second material includes graphene.

3. The bioelectrode according to claim 2, wherein:
the first channel is formed on the substrate,
the second channel is formed on the first channel, and
the first channel is disposed between the substrate and the second channel.

4. The bioelectrode according to claim 2, wherein:
the second channel is formed on the substrate,
the first channel is formed on the second channel, and
the second channel is disposed between the substrate and the first channel.

5. The bioelectrode according to claim 1, wherein the constituent material of the substrate includes parylene-C.

6. An electrode sensor, comprising:
a substrate;
an electrode part formed on the substrate; and
a bio-receptor formed on the electrode part,
wherein the electrode part includes a channel having a bilayer structure of a first channel including a first material and a second channel including a second material different from the first material, and
wherein the first material and the second material include a biocompatible material.

7. The electrode sensor according to claim 6, wherein the bio-receptor includes an aptamer capable of binding to a predefined target molecule.

8. The electrode sensor according to claim 7, wherein the target molecule includes a cortisol molecule.

9. The electrode sensor according to claim 6, wherein the bio-receptor is fixed to the channel of the electrode part.

10. An electrode sensor, comprising:
a bioelectrode; and
a measuring device linked to the bioelectrode and configured to constantly measure a biosignal based on a sensing value from the bioelectrode,
wherein the bioelectrode includes:
a substrate; and
an electrode part formed on the substrate,
wherein the electrode part includes a channel having a bilayer structure of a first channel including a first material and a second channel including a second material different from the first material,
wherein the first material and the second material include a biocompatible material, and
wherein the biosignal includes at least one of electrocardiography (ECG), electroencephalography (EEG), and electromyography (EMG).
